(19) United States
(12) Patent Application Publication
(10) Pub. No.: US 2025/0144654 A1
(43) Pub. Date: May 8, 2025

(54) TRIGGER ASSEMBLY FOR ATOMIZER AND ATOMIZER

(71) Applicant: Atsenbo (Suzhou) Pharmaceutical Technology Co., Ltd, Suzhou City, Jiangsu Province (CN)

(72) Inventor: Hongxian REN, Suzhou City, Jiangsu Province (CN)

(21) Appl. No.: 18/838,066

(22) PCT Filed: Feb. 20, 2023
(86) PCT No.: PCT/CN2023/077212

§ 371 Date: Aug. 13, 2024

(30) Foreign Application Priority Data
Mar. 8, 2022 (CN) 202210220882.7

(51) Int. Cl. B05B 11/10; A61M 15/00; B05B 12/00

(52) U.S. Cl. CPC B05B 11/1091; A61M 15/0001; B05B 12/0026

(57) ABSTRACT

Provided is a trigger assembly for an atomizer and an atomizer that includes the trigger assembly. The trigger assembly includes a trigger ring and a delivery tube holder. The trigger assembly has a locked position and an unlocked position. In the locked position, the trigger ring stops axial movement of the delivery tube holder, and in the unlocked position, the delivery tube holder can move in an axial direction relative to the trigger ring. The trigger assembly further includes an actuation member. The actuation member is actuated to drive the delivery tube holder to pivot and drive the trigger ring to move, so that the trigger assembly is switched between the locked position and the unlocked position.

### TECHNICAL FIELD

The present disclosure relates to a trigger assembly for an atomizer, and an atomizer. The atomizer may be used, for example, for atomizing and/or spraying a liquid medicine.

### BACKGROUND ART

In the related art, a container of an atomizer (or referred to as a sprayer) contains a liquid to be atomized or sprayed, and during movement of the container relative to a spraying assembly (such as a pump), the liquid in the container is atomized and the atomized liquid is sprayed from a nozzle of the spraying assembly. However, operations of such an atomizer are usually not stable or reliable enough. For example, when it is necessary for a user to perform more than one operation step (such as one rotation operation and one pressing operation) to actuate the atomizer, a misoperation is likely to occur. This misoperation of the atomizer is undesired, which not only increases the difficulty of use, but may also lead to damage or failure of the atomizer.

### SUMMARY OF THE INVENTION

An objective of the present disclosure is to provide a trigger assembly for an atomizer. The trigger assembly provides multiple unlocking modes, thus effectively preventing a misoperation of the trigger assembly.

According to a first aspect of the present disclosure, a trigger assembly for an atomizer is provided. Herein, the trigger assembly comprises a trigger ring and a delivery tube holder, wherein the trigger assembly has a locked position and an unlocked position. In the locked position, the trigger ring stops axial movement of the delivery tube holder, and in the unlocked position, the delivery tube holder can move in an axial direction relative to the trigger ring. The trigger assembly further comprises an actuation member. The actuation member can be actuated to drive the delivery tube holder to pivot and drive the trigger ring to move, so that the trigger assembly is switched between the locked position and the unlocked position.

In the trigger assembly according to the present disclosure, two unlocking modes of the atomizer can be implemented, so that the problem of the atomizer being prone to a misoperation when only one unlocking mode is provided can be solved, and therefore damage to the trigger assembly caused by the misoperation by a user can be prevented. In addition, the trigger assembly is user-friendly, because the user does not have to remember whether his/her operation steps are correct, but has a choice of multiple unlocking modes. In addition, the trigger assembly not only reduces the use difficulty of the atomizer, but also prevents excessive interference between components, thus prolonging the service life of the trigger assembly.

In an implementation, the actuation member comprises an actuation member body and an actuation protrusion axially protruding from the actuation member body, the actuation protrusion being configured to drive the trigger ring to move from the locked position to the unlocked position, and/or to drive the trigger ring to move from the unlocked position to the locked position.

Preferably, the actuation member comprises a plurality of actuation protrusions, wherein at least one actuation protrusion of the plurality of actuation protrusions is configured to drive the trigger ring to move from the locked position to the unlocked position, and at least another actuation protrusion of the plurality of actuation protrusions is configured to drive the trigger ring to move from the unlocked position to the locked position.

Preferably, the actuation member comprises a plurality of actuation protrusions, wherein each of the plurality of actuation protrusions can drive the trigger ring to move from the locked position to the unlocked position, and can drive the trigger ring to move from the unlocked position to the locked position.

Preferably, the plurality of actuation protrusions are arranged at equal angular intervals on the actuation member body in a circumferential direction of the actuation member.

Preferably, an outer peripheral wall of the actuation protrusion extends from a peripheral wall of the actuation member or forms a part of a peripheral wall of the actuation member.

Preferably, at least one side wall of the actuation protrusion is configured to guide movement of the trigger ring.

Preferably, an angle between the at least one side wall of the actuation protrusion for guidance and the outer peripheral wall is less than 45°, and the angle is preferably in a range of 25° to 45°, preferably in a range of 30° to 40°, and particularly preferably in a range of 31° to 35°.

Preferably, at least one side wall of the actuation protrusion is configured to stop the movement of the trigger ring.

Preferably, an angle between the at least one side wall of the actuation protrusion for stopping and the outer peripheral wall is greater than 60°, and the angle is preferably in a range of 60° to 80°, preferably in a range of 65° to 70°, and particularly preferably in a range of 68° to 75°.

Preferably, a length or an arc length of the at least one side wall of the actuation protrusion for guidance on a horizontal projection plane is greater than or equal to a length or an arc length of the at least one side wall of the actuation protrusion for stopping on the horizontal projection plane.

Preferably, the outer peripheral wall and/or the side walls of the actuation protrusion are in the form of a flat surface, a curved surface or a combination of a flat surface and a curved surface.

Preferably, the trigger ring comprises a ring body and a protruding section axially protruding from the ring body.

Preferably, a side surface of the actuation protrusion can abut against a side surface of the protruding section to move the trigger ring from the locked position to the unlocked position and/or from the unlocked position to the locked position.

Preferably, the side wall of the protruding section is in the form of a flat surface, a curved surface or a combination of a flat surface and a curved surface.

Preferably, the delivery tube holder is sleeved in the actuation member so as to pivot together with the actuation member.

Preferably, the actuation member comprises a rib arranged on an inner wall of the actuation member body, and the delivery tube holder comprises a groove provided on an outer wall of the tube holder body.

Preferably, in the locked position, the trigger ring is pressed against the delivery tube holder and the actuation member, and in the unlocked position, the trigger ring is pressed against only the actuation member, but is not pressed against the delivery tube holder.

Preferably, a width of the groove is greater than or approximately equal to a width of the rib, so that the delivery tube holder can move relative to the actuation member in an axial direction, but cannot move relative to the actuation member in a circumferential direction.

Preferably, in the locked position, the trigger ring is arranged non-coaxially with respect to the delivery tube holder and the actuation member, and in the unlocked position, the trigger ring is arranged coaxially with respect to the delivery tube holder and the actuation member.

Preferably, the trigger ring comprises a ring body and a first locking portion arranged on the ring body, and the delivery tube holder comprises a tube holder body and a second locking portion arranged on the tube holder body.

Preferably, in the locked position, the first locking portion abuts against the second locking portion, so that the trigger ring stops the axial movement of the delivery tube holder.

Preferably, in the locked position, the first locking portion abuts against the second locking portion, so that the trigger ring and the delivery tube holder stop each other.

Preferably, the trigger ring and the delivery tube holder stopping each other consists in that the trigger ring prevents a vertical movement of the delivery tube holder, and the delivery tube holder prevents a horizontal movement of the trigger ring.

Preferably, the first locking portion is in the form of a locking protrusion extending out from at least one side of the trigger ring, and the second locking portion is in the form of a locking notch recessed on at least one side of the tube holder body.

Preferably, the first locking portion is in the form of a locking notch recessed on at least one side of the trigger ring, and the second locking portion is in the form of a locking protrusion extending out from at least one side of the tube holder body.

Preferably, an outer diameter of the delivery tube holder is less than or equal to an inner diameter of the trigger ring.

Preferably, only one locking protrusion is provided on at least one side of the trigger ring, and a plurality of locking notches are provided on at least one side of the delivery tube holder.

Preferably, the plurality of locking notches are arranged at equal angular intervals in a circumferential direction on a side of the delivery tube holder that mates with the locking protrusion.

Preferably, the first locking portion is in the form of a first locking surface, and the second locking portion is in the form of a second locking surface.

Preferably, at least one of the first locking surface and the second locking surface is in the form of a flat inclined surface, and the inclined surface is inclined with respect to both a horizontal direction and a vertical direction.

Preferably, the first locking surface forms a first inclined locking surface, and the second locking surface forms a second inclined locking surface.

Preferably, the delivery tube holder further comprises an inclined guide surface on a side where the locking notch is provided, and the inclined guide surface has an edge shared with the second locking surface.

According to a second aspect of the present disclosure, an atomizer is provided. Herein, the atomizer comprises a trigger assembly as described above, for triggering the atomizer to spray an atomized liquid. Therefore, even if an operation of the atomizer requires more than one step, damage to the atomizer caused by a misoperation of a user is avoided.

Preferably, the actuation member and/or the delivery tube holder is in the form of a housing of the atomizer; or at least one of the actuation member and the delivery tube holder is connected to the housing of the atomizer, so that the actuation member and the delivery tube holder can rotate as the housing rotates.

Preferably, the trigger ring is in the form of a switch button of the atomizer; or the trigger ring is connected to a switch button of the atomizer, so that the trigger ring can move as the switch button is pressed.

Preferably, when the atomizer is in an initial position, the trigger assembly is located in the unlocked position; and

when the atomizer is in a pre-trigger position, the trigger assembly is located in the locked position.

Preferably, the atomizer further comprises a spring at a bottom of the delivery tube holder, for applying a thrust to the delivery tube holder to urge the delivery tube holder to pass through the trigger ring in an axial direction.

Preferably, the atomizer further comprises: a container configured to contain a liquid to be atomized; and a spraying assembly configured to atomize the liquid sucked from the container and spray the atomized liquid.

### BRIEF DESCRIPTION OF THE DRAWINGS

In order to more clearly describe the technical solutions in the embodiments of the present disclosure or in the prior art, the accompanying drawings required for describing the embodiments or the prior art are briefly described below. Apparently, the accompanying drawings in the following description merely show some of the embodiments of the present disclosure, and those of ordinary skill in the art may still derive other accompanying drawings according to the structures shown by these accompanying drawings without creative efforts. The figures are as follows:

FIG. 1 is a front view showing a trigger ring according to an implementation of the present disclosure;

FIG. 2 is a top view showing a trigger ring according to an implementation of the present disclosure;

FIG. 3 is a partially enlarged view showing the trigger ring shown in FIG. 2;

FIG. 4 is a perspective view showing a trigger ring according to an implementation of the present disclosure;

FIG. 5 is a front view showing a delivery tube holder according to an implementation of the present disclosure;

FIG. 6 is a top view showing a delivery tube holder according to an implementation of the present disclosure;

FIG. 7 is a partially enlarged view showing the delivery tube holder shown in FIG. 6;

FIG. 8 is a perspective view showing a delivery tube holder according to an implementation of the present disclosure;

FIG. 9 is a front view showing an actuation member according to an implementation of the present disclosure;

FIG. 10 is a side view showing an actuation member according to an implementation of the present disclosure;

FIG. 11 is a top view showing an actuation member according to an implementation of the present disclosure;

FIG. 12 is a perspective view showing an actuation member according to an implementation of the present disclosure;

FIG. 13 is a front view showing a trigger assembly located in a locked position according to an implementation of the present disclosure;

FIG. 14 is a side view showing a trigger assembly located in a locked position according to an implementation of the present disclosure;

FIG. 15 is a top view taken along line C-C of FIG. 14 and showing a trigger assembly located in a locked position according to an implementation of the present disclosure;

FIG. 16 is a front view showing a trigger assembly located in another locked position according to an implementation of the present disclosure;

FIG. 17 is a side view showing a trigger assembly located in another locked position according to an implementation of the present disclosure;

FIG. 18 is a top view taken along line C-C of FIG. 17 and showing a trigger assembly located in another locked position according to an implementation of the present disclosure;

FIG. 19 is a front view showing a trigger assembly located in an unlocked position according to an implementation of the present disclosure;

FIG. 20 is a side view showing a trigger assembly located in an unlocked position according to an implementation of the present disclosure;

FIG. 21 is a top view taken along line C-C of FIG. 20 and showing a trigger assembly located in an unlocked position according to an implementation of the present disclosure;

FIG. 22 is an exploded view showing an atomizer according to an implementation of the present disclosure;

FIG. 23 is a schematic view showing an atomizer according to an implementation of the present disclosure; and

FIG. 24 is a sectional view showing the atomizer of FIG. 22 taken along line C-C.

### DETAILED DESCRIPTION OF EMBODIMENTS

The technical solutions in the embodiments of the present disclosure are clearly and completely described below with reference to the accompanying drawings for the embodiments of the present disclosure. Apparently, the described embodiments are merely some rather than all of the embodiments of the present disclosure. Based on the embodiments of the present disclosure, all other embodiments obtained by those of ordinary skill in the art without creative efforts fall within the scope of protection of the present disclosure.

It should be noted that all directional indications (such as up, down, left, right, front, and back) in the embodiments of the present disclosure are only intended to explain a relative positional relationship, a movement status, and the like between components in a specific posture (as shown in the figures). If the specific posture changes, the directional indications change accordingly.

In the present disclosure, unless otherwise clearly specified and limited, the terms such as “connection” and “fixing” should be understood in a board sense, which, for example, may be a direct connection or an indirect connection implemented by means of an intermediate medium, or may be internal communication between two elements or an interaction relationship between two elements, unless otherwise clearly limited. For those of ordinary skill in the art, specific meanings of the foregoing terms in the present disclosure may be understood based on specific situations.

In the present disclosure, unless otherwise specified, all numbers expressing component parameters, technical effects, etc. used in this description and claims should be understood as being modified by the term “about” or “approximately” in any case. Therefore, unless indicated to the contrary, numerical parameters as set forth in the following description and appended claims are approximations. For those skilled in the art, each numerical parameter may vary depending upon the desired properties and effects sought to be obtained by the present disclosure and should be construed in light of the number of significant digits and conventional rounding methods or in a manner understood by those skilled in the art.

The terms used in the description of the various examples in the present disclosure are for the purpose of describing particular examples only and are not intended to be limiting. Unless the context clearly indicates otherwise, if the number of elements is not specifically limited, there may be one or more elements. Moreover, the term “and/or” used in the present disclosure encompasses any of and all possible combinations of listed items.

In the scope of the present disclosure, an “atomizer” (also referred to as a “sprayer”) refers to a device for atomizing a liquid. Generally, the atomizer is configured to atomize a fluid (e.g., a liquid medicine or the like) and spray the atomized fluid to some parts of a user (such as a patient) to be treated. Since the liquid medicine is loaded in the atomizer, the stability and operation reliability of the atomizer are particularly important.

In the related art, the atomizer may be operated by mechanical means, such as a push switch or a rotary switch. However, the use of the atomizer may not be friendly enough to a user, and especially when the user needs to perform a plurality of operations to complete atomizing and spraying, a misoperation is likely to occur. For example, when the atomizer is configured such that the user needs to perform two operations, such as rotating a housing and pressing a switch, to complete one spraying, the user may forget to press the switch after the rotating operation, resulting in another rotating operation during the next use. This misoperation may cause components of the atomizer to interfere with or squeeze each other. In addition, for example, when the user performs some operations but does not complete all operations, the atomizer is in a pre-trigger state, which is not stable enough. If, in this case, the atomizer is subjected to some impacts or shaking, or may even accidentally slipping from a high level, these accidents may cause relative movement of components of the atomizer and therefore undesired atomization of the liquid.

In view of this, the present disclosure provides an improved trigger assembly, which is mounted in an atomizer, and preferably can be linked with a push switch and/or a rotary switch of the atomizer. Herein, reliable switching of the trigger assembly between the locked position and the unlocked position is implemented under the action of an actuation member that can actuate a delivery tube holder and a trigger ring.

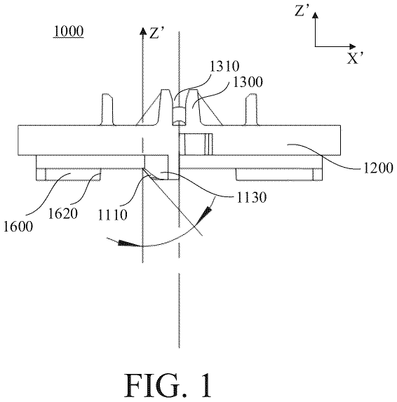

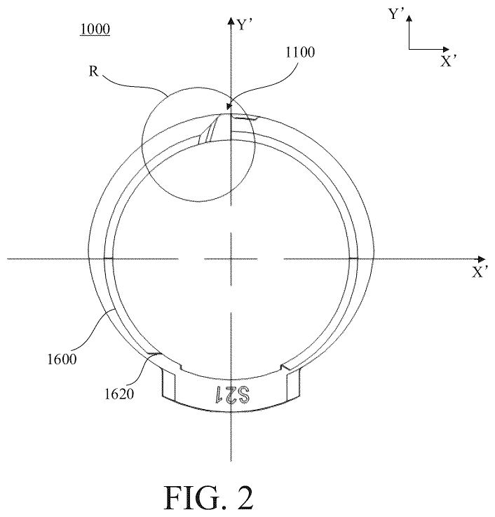

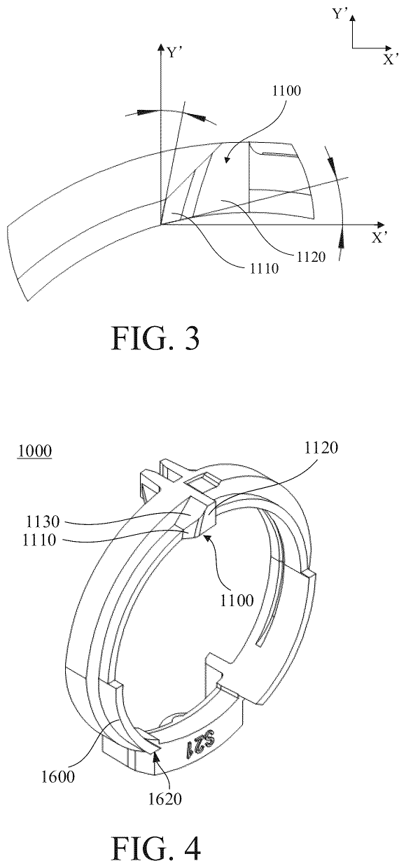

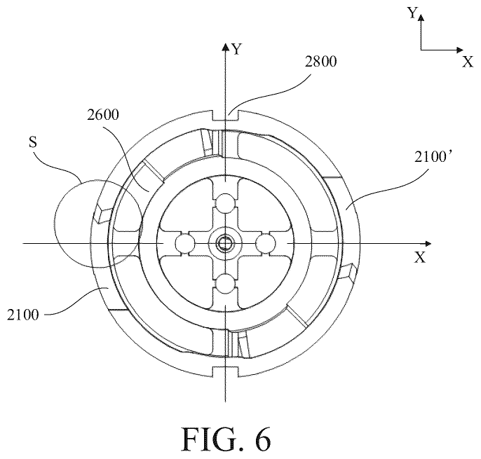

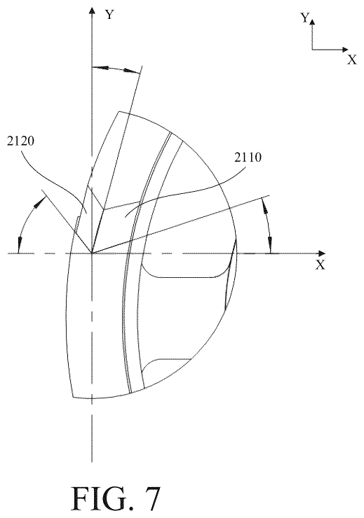

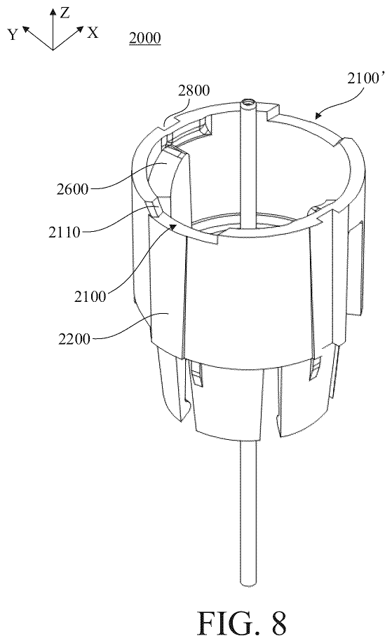

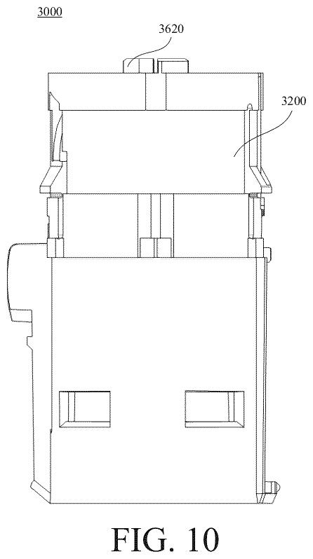

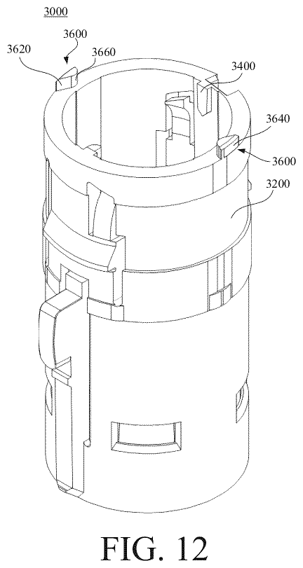

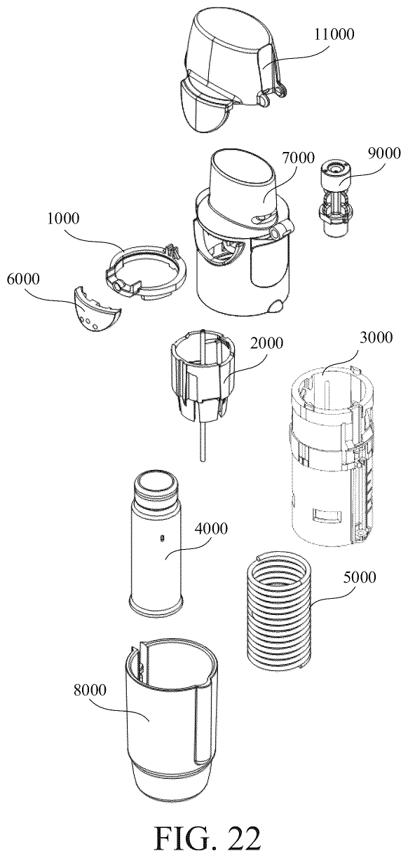

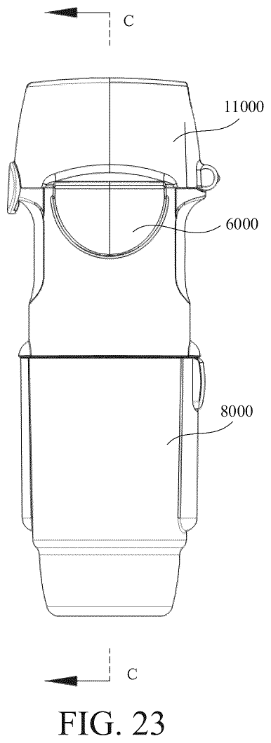

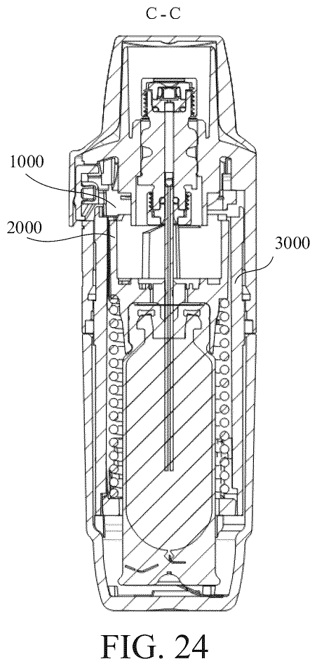

In the scope of the present disclosure, the “trigger assembly” refers to an assembly configured to control triggering of the atomizer, such as an assembly that can control and/prevent an atomization or spraying operation that is performed by the atomizer. In the present disclosure, the trigger assembly may comprise, for example, a trigger ring 1000, a delivery tube holder 2000, and an actuation member 3000, with specific structures described in detail below with reference to FIGS. 1 to 4, FIGS. 5 to 8, and FIGS. 9 to 12, respectively. In addition, the arrangement of the trigger assembly in the atomizer may be described in detail, for example, with reference to FIGS. 22 to 24.

It should be understood that the trigger assembly may comprise not only the trigger ring 1000, the delivery tube holder 2000, and the actuation member 3000, but may also comprise other elements, provided that a function of controlling and/or preventing triggering can be achieved. Herein, importantly, the trigger assembly can be switched between (one or more) locked positions and (one or more) unlocked positions by means of an interaction between the trigger ring 1000, the delivery tube holder 2000, and the actuation member 3000. Herein, the “interaction” consists in that, for example, the actuation member 3000 can not only be actuated to drive the delivery tube holder 2000 to pivot, but can also push or drive the trigger ring 1000 to move. In a preferred implementation, the actuation member 3000 can not only be actuated to drive the delivery tube holder 2000 or the trigger ring 1000 to pivot or move from a locked position to an unlocked position, but can also be actuated to drive the delivery tube holder 2000 or the trigger ring 1000 to pivot or move from the unlocked position to the locked position.

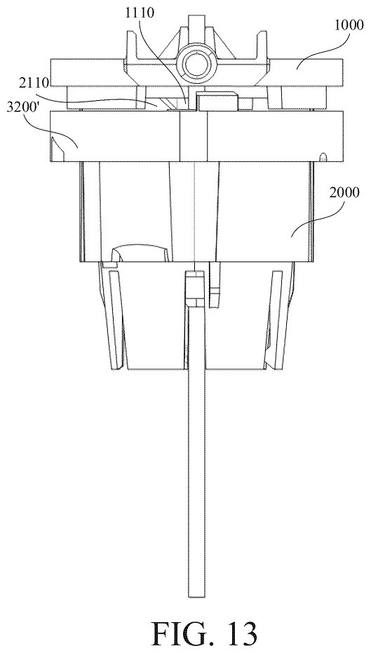

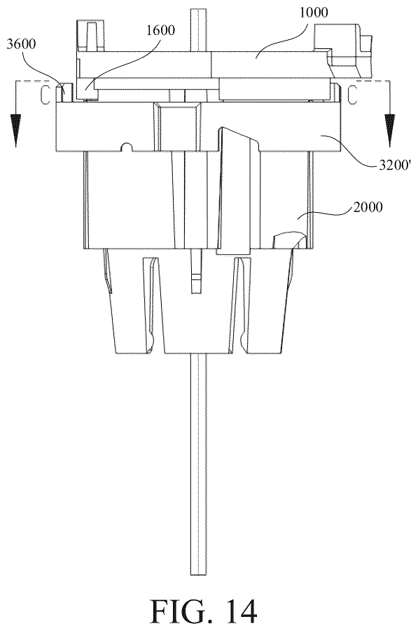

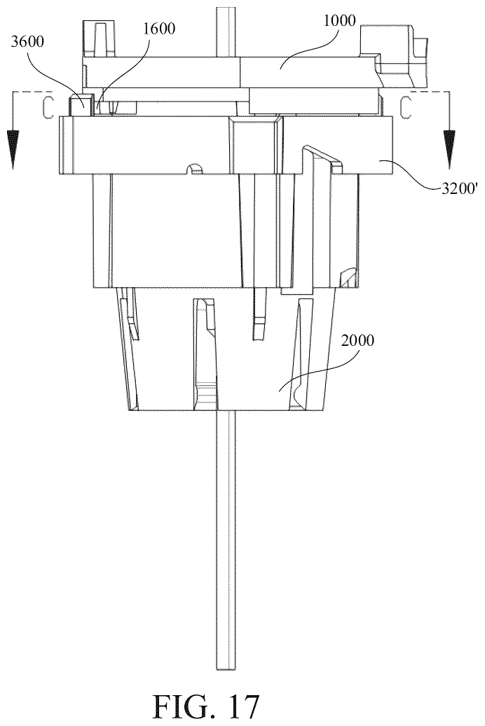

In the scope of the present disclosure, the “locked position” of the trigger assembly refers to a position where the atomizer is locked, that is, a position where the atomizer cannot operate without human intervention. In other words, when the trigger assembly is in a “locked position”, the atomizer can perform an atomization or spraying operation only after manual operation. This operation may be to rotate a component and/or push a switch, etc. In the present disclosure, the “locked position” of the trigger assembly may alternatively refer to a position where the trigger ring 1000 achieves an effect of stopping the delivery tube holder 2000, that is, the delivery tube holder 2000 is stopped in this locked position and cannot further move axially. As a preferred example, the “locked position” may comprise a position where the trigger ring 1000 and the delivery tube holder 2000 stop each other. Herein, “stopping each other” may mean, for example, that the trigger ring 1000 prevents a movement, such as a vertical movement, of the delivery tube holder 2000, while the delivery tube holder 2000 also prevents a further movement, such as a horizontal and/or vertical translation, of the trigger ring 1000. The “locked position” is further described below by virtue of FIGS. 13 to 15 and FIGS. 16 to 18.

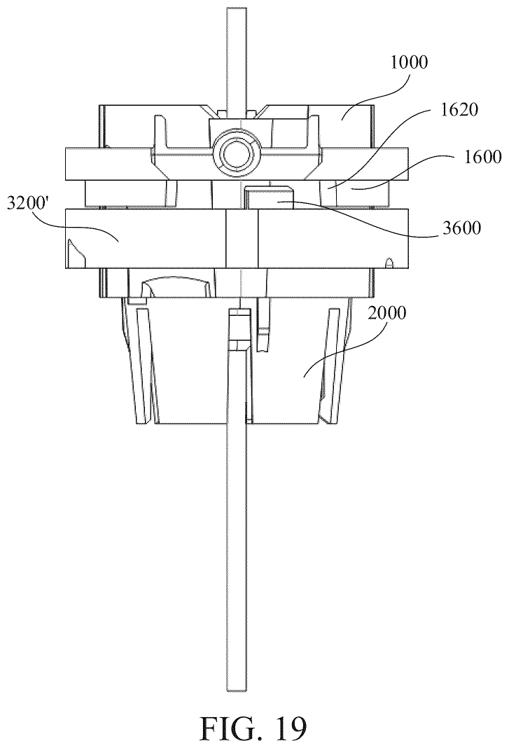

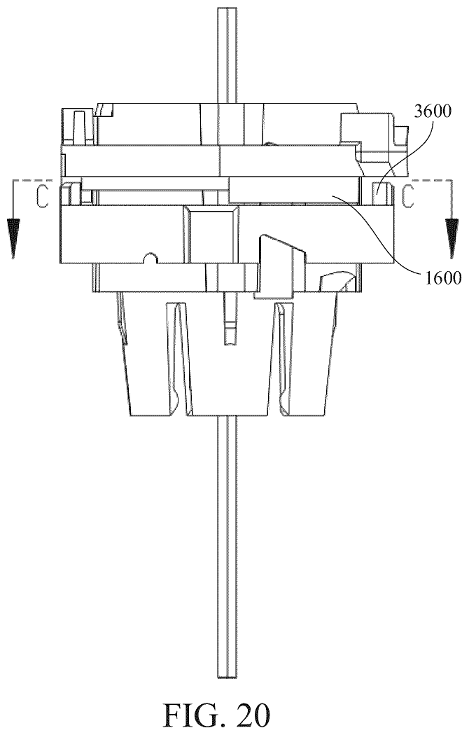

In the scope of the present disclosure, the “unlocked position” of the trigger assembly refers to a position where the atomizer is not locked, that is, an operable position of the atomizer. In other words, when the trigger assembly is in the “unlocked position”, components (such as the trigger ring 1000 and the delivery tube holder 2000) in the atomizer can move relative to each other, so as to achieve an effect of atomizing the liquid and/or spraying the atomized liquid. It may be understood that the “unlocked position” of the trigger assembly is a concept with respect to the “locked position” described above. As long as the atomizer is not locked, the trigger assembly is located in the unlocked position. Therefore, the trigger assembly may, for example, have a plurality of “unlocked positions”, and the trigger assembly may be switched between the locked position and an unlocked position, or between a plurality of unlocked positions. During switching of the trigger assembly, the atomizer can be switched between an initial position, a pre-trigger position, and a triggered position. The “unlocked position” is further described below by means of FIGS. 19 to 21.

First, a trigger ring 1000 according to an implementation of the present disclosure is described with reference to FIGS. 1 to 4. The trigger ring 1000 may comprise a ring body 1200 and a first locking portion (herein, for example, a locking protrusion 1100). The locking protrusion 1100 protrudes downwardly from a bottom side of the ring body 1200.

It may be understood that although only one locking protrusion 1100 protruding downwardly from the bottom side of the ring body 1200 is shown in the figures, the number and arrangement of locking protrusions 1100 are not limited thereto. As an example, the trigger ring 1000 may alternatively comprise a plurality of locking protrusions 1100. For example, two or three locking protrusions 1100 are arranged on a side of the trigger ring, and these locking protrusions 1100 are preferably circumferentially arranged at equal angular intervals in an array, so that multi-stage locking and unlocking of the trigger assembly can be achieved, for example. For example, when two locking protrusions 1100 are provided, the two locking protrusions 1100 are arranged at an angular interval of 180° in an array along a circumference of the ring body 1200.

As another example, it is also possible that locking protrusions 1100 are arranged on two sides of the ring body 1200. In this case, the locking protrusions 1100 on an upper side and a lower side are staggered from each other, for example, and respectively mate, and are linked, with locking notches 2100 on the upper side and the lower side of the trigger ring 1000.

In addition, the trigger ring 1000 further comprises a protruding section 1600 protruding downwardly from the bottom side of the ring body 1200, but the number and arrangement of protruding sections 1600 are not limited thereto. As an example, the trigger ring 1000 may comprise two, three or more protruding sections 1600, and these locking protrusion portions 1100 are preferably circumferentially arranged at equal angular intervals in an array or arranged in a mirrored manner. As can be seen from the top view, the protruding section 1600 may be an arc-shaped section extending along a periphery of the ring body 1200. The arc-shaped section preferably comprises a central angle of 30° to 50°, particularly preferably 35° to 45°.

The protruding section 1600 may comprise a side wall 1620 configured to be actuated by the actuation protrusion 3600 of the actuation member 3000. The side wall 1620 may be a flat surface or a curved surface, preferably a flat surface or a curved surface that can be complementary to a corresponding actuation surface of the actuation protrusion 3600.

It can be further seen in the figures that some rib structures are respectively provided on the two sides of the ring body 1200 for mutual connection or mating with other components. For example, two ribs 1300 on top of the ring body 1200 form a slot 1310. The slot can mate with a rotating member of the atomizer, so as to limit rotation of the ring body 1200 to make the ring body 1200 only translate. Herein, the translation of the ring body 1200 can be implemented by the actuation protrusion 3600 of the actuation member 3000. In a preferred implementation, in a spraying stroke, an actuation protrusion 3600 is configured to drive or push the trigger ring 1000 to move from the locked position to the unlocked position (in a Y′ direction), while another actuation protrusion 3600 is configured to drive or push the trigger ring to move from the unlocked position to the locked position (in a −Y′ direction).

The locking protrusion 1100 may comprise a first locking surface 1110, which is, for example, a lower inclined surface of the locking protrusion 1100. Preferably, the first locking surface 1110 is an inclined surface that is inclined in three-dimensional directions, that is, the first locking surface 1110 is not only inclined relative to a horizontal plane (an X′Y′ plane), but also inclined relative to vertical planes (a Z′Y′ plane and an X′Z′ plane). In other words, the first locking surface 1110 is both a radially outward surface of the trigger ring 1000 and an axially outward surface of the trigger ring 1000. Herein, this three-dimensionally inclined arrangement of the first locking surface 1110 is very advantageous, and thus the three-dimensionally inclined first locking surface 1110 achieves a desired balance effect between prevention of being stuck and prevention of automatic atomization.

The locking protrusion 1100 further comprises a guide surface 1120, which is herein a bottom surface of the locking protrusion 1100 and is horizontally arranged. Of course, the guide surface 1120 may be alternatively arranged slightly inclined with respect to the horizontal plane. During position switching of the trigger assembly, the guide surface 1120 may be in contact with a groove bottom of the locking notch 2100, thus making the switching process smoother.

In addition, the locking protrusion 1100 further comprises a transition surface 1130 configured to limit rotational movement of the trigger ring 1000, so that the trigger ring 1000 can only translate under the constraint of the transition surface.

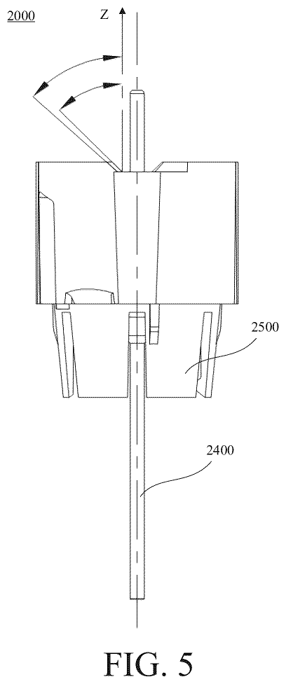

A delivery tube holder 2000 according to an implementation of the present disclosure is described in detail below with reference to FIGS. 5 to 8. The delivery tube holder 2000 may comprise a barrel-shaped tube holder body 2200 and a second locking portion (herein, for example, a locking notch 2100 recessed on a side of the tube holder body 2200) arranged on the tube holder body. Although two locking notches 2100 and 2100′ are shown in the figures, it should be understood that the number and arrangement of locking notches 2100 are not restrictive, but any number of locking notches 2100 may be provided on an upper wall of the tube holder body 2200, and a different number of second locking surfaces 2110 may be provided on a wall of one or either side of each of the locking notches 2100. The possibility of flexible arrangement of the locking notches 2100 enables a motion level of the unlocking assembly to be adapted as required.

In addition, the delivery tube holder 2000 further comprises a groove 2800 recessed from an outer wall of the tube holder body 2200. The groove 2800 is configured to receive a rib 3400 of the actuation member 3000 that protrudes inwardly from an inner wall of the actuation member body. When the rib 3400 of the actuation member 3000 is received in the groove 2800 of the delivery tube holder 2000, the actuation member 3000 can be actuated to drive the delivery tube holder 2000 to pivot together. It should be understood that due to the interaction between the groove 2800 and the rib 3400, the pivoting movement of the delivery tube holder 2000 may be restricted by the actuation member 3000 and rotate due to the rotation of the actuation member 3000, but the axial movement of the delivery tube holder 2000 may be restricted by the trigger ring 1000. It should be understood that although two grooves 2800 and two corresponding ribs 3400 are shown in the figures and are circumferentially arranged at equal angular intervals, the number and arrangement of grooves 2800 and ribs 3400 are not limited thereto.

A width of the groove 2800 of the delivery tube holder 2000 may be greater than or approximately equal to a sum of widths of the ribs 3400 of the actuation member 3000, so as to restrict relative rotation between the delivery tube holder 2000 and the actuation member 3000 in a circumferential direction, but not to restrict axial relative movement between the delivery tube holder 2000 and the actuation member 3000 in an axial direction. Herein, guidance of the axial relative movement is important.

At least one wall of the locking notch 2100 forms a second locking surface 2110, and the second locking surface 2110 mates with the first locking surface 1110 described above. That is, when the trigger assembly is located in the locked position, the second locking surface 2110 and the first locking surface 1110 abut against each other. As the trigger ring 1000 moves, for example, under the action of the push switch, the first locking surface 1110 abuts against the second locking surface 2110 and slides thereon, and when the first locking surface slides past the second locking surface 2110, the trigger assembly enters the unlocked position.

Similar to the first locking surface 1110, the second locking surface 2110 is also a three-dimensionally inclined surface, that is, the second locking surface 2110 is not only inclined relative to a horizontal plane (an XY plane), but also inclined relative to vertical planes (a ZY plane and an XZ plane). In other words, the second locking surface 2110 is both a radially inward surface of the delivery tube holder 2000 and an axially inward surface of the delivery tube holder 2000.

Herein, this three-dimensionally inclined arrangement of the second locking surface 2110 is very advantageous, and thus a desired balance effect between prevention of being stuck and prevention of automatic atomization is achieved.

The locking notch 2100 may comprise a groove bottom and two side walls, and the second locking surface 2110 is provided in one wall. As can also be seen from the figure, in a preferred embodiment, the locking notch 2100 further comprises an inclined guide surface 2120, the inclined guide surface 2120 has an edge shared with the second locking surface 2110, and a sum of a radial dimension d1 of the second locking surface 2110 and a radial dimension of the inclined guide surface 2120 is equal to a radial wall thickness dimension of the tube holder body 2200 on this side. The inclined guide surface 2120 also forms a substantially parallelogram shape and is substantially arranged at the same position with the same angle in the circumferential direction as the second locking surface 2110. It may be understood that the inclined guide surface 2120 is used as only a preferred arrangement structure, but is not necessary. When no inclined guide surface 2120 is provided, the second locking surface 2110 may account for the entire radial dimension of the wall thickness.

The delivery tube holder 2000 further comprises a connecting piece 2500 that is connected to other members of the sprayer, such as a container 4000 or a housing 8000, for example, by snap-fit connection. The delivery tube holder 2000 further comprises a delivery tube 2400 configured to deliver a liquid stored in the container 4000 to a spray chamber for spraying.

In a preferred implementation, an elastic member, such as a spring 5000, may be further arranged at a bottom of the delivery tube holder 2000. The spring 5000 always applies an upward thrust to the delivery tube holder 2000, so that once the trigger assembly leaves the locked position, the delivery tube holder 2000 can move upwardly, that is, move axially. When an outer diameter of the delivery tube holder 2000 is less than or equal to an inner diameter of the trigger ring 1000, the delivery tube holder 2000 can pass through the trigger ring 1000 upwardly under the action of the spring 5000, and when a downward force is applied to the delivery tube holder 2000, the delivery tube holder 2000 can move downwardly against the elastic force and return to the locked position. It should be understood that the spring 5000 is a preferred component of the atomizer, but is not necessary. Other alternative components, such as buttons, are also conceivable provided that these components can also provide an axial upward thrust for the delivery tube holder 2000.

The delivery tube holder 2000 may be further internally provided with a spiral portion 2600, which is arranged on an upper surface of a protruding portion radially inward from an inner wall of the tube holder body 2200. When the delivery tube holder 2000 is arranged in the atomizer, the spiral portion 2600 may, for example, mate with a corresponding spiral portion of a mouthpiece 7000. Therefore, when the delivery tube holder 2000 is rotated, the delivery tube holder 2000 can move in the vertical direction, that is, in the axial direction. In the present disclosure, during downward movement of the delivery tube holder 2000, the delivery tube holder 2000 performs downward rotary movement under the action of the spiral portion, that is, performs a composite motion of rotation and downward translation, while during upward movement of the delivery tube holder 2000, the delivery tube holder 2000 can perform a simple upward translation movement without any rotation.

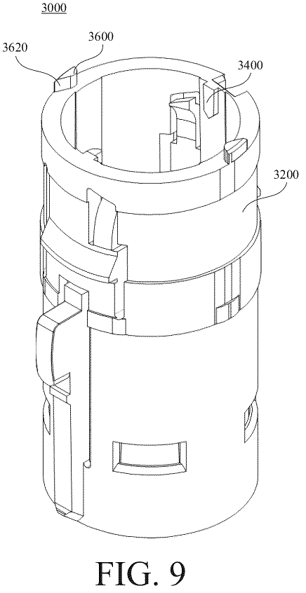

An actuation member 3000 according to an implementation of the present disclosure is described in detail below with reference to FIGS. 9 to 12.

In order to switch the trigger assembly between the locked position and the unlocked position, the actuation of the actuation member 3000 comprises not only actuation to the delivery tube holder 2000 (for example, driving the delivery tube holder 2000 to pivot), but also actuation to the trigger ring 1000 (for example, driving the trigger ring 1000 to move).

The actuation member 3000 may comprise an actuation member body 3200 and an actuation protrusion 3600. The actuation protrusion 3600 axially protrudes from the actuation member body 3200, and is configured to drive the trigger ring 1000 to move from the locked position to the unlocked position, and/or to drive the trigger ring 1000 to move from the unlocked position to the locked position. In a preferred implementation, the actuation member 3000 may comprise a plurality of actuation protrusions 3600 (for example, two actuation protrusions 3600 shown in the figures). One of the actuation protrusions 3600 is configured to drive the protruding section 1600 of the trigger ring 1000 to move the trigger ring from the locked position to the unlocked position, and the other actuation protrusion 3600 is configured to drive the protruding section 1600 of the trigger ring 1000 to move the trigger ring from the unlocked position to the locked position.

In a preferred implementation, the actuation protrusion 3600 may actuate the trigger ring 1000 in two directions, that is, the same actuation protrusion 3600 can drive the trigger ring 1000 to move from the locked position to the unlocked position, and can also drive the trigger ring 1000 to move from the unlocked position to the locked position. It may be understood that when, for example, two actuation protrusions 3600 are provided, the actuation protrusions 3600 may alternately actuate the trigger ring 1000, that is, directions in which the actuation protrusions drive the trigger ring 1000 may be interchanged. As an example, in a stroke (for example, corresponding to an atomization and spraying of the atomizer), a first actuation protrusion is used to drive the trigger ring 1000 to move from the locked position to the unlocked position, and a second actuation protrusion is used to drive the trigger ring 1000 to move from the unlocked position to the locked position, while in a next stroke, the first actuation protrusion is used to drive the trigger ring 1000 to move from the unlocked position to the locked position, and the second actuation protrusion is used to drive the trigger ring 1000 to move from the locked position to the unlocked position. Therefore, the actions of the plurality of actuation protrusions 3600 can be interchanged.

When the plurality of actuation protrusions 3600 are provided, preferably, the actuation protrusions 3600 are arranged at equal angular intervals on the actuation member body 3200 in a circumferential direction of the actuation member 3000. As shown, two actuation protrusions 3600 are arranged at an angular interval of 180° on a circumference of the actuation member body 3200.

The actuation protrusion 3600 may comprise an outer peripheral wall 3640, which extends from a peripheral wall of the actuation member 3000 or forms a part of a peripheral wall of the actuation member 3000.

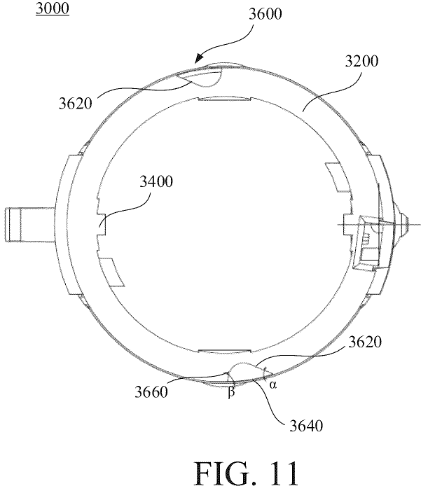

The actuation protrusion 3600 may comprise a side wall 3620. The side wall 3620 is configured to actuate the side wall 1620 of the protruding section 1600 of the trigger ring 1000, or in other words to guide the trigger ring 1000 to move. The side wall 3620 may be in the form of a flat surface or a curved surface or a combination of a flat surface and a curved surface, and preferably in the form of a surface mating with or complementary to the side wall 1620 of the protruding section 1600. As shown in FIG. 11, an angle α between the side wall 3620 and the outer peripheral wall 3640 is less than 45°, and the angle α is preferably in a range of 25° to 45°, preferably in a range of 30° to 40°, and particularly preferably in a range of 31° to 35°. With the arrangement of the side wall 3620 and/or the setting of the angle α, the side wall 3620 can make the protruding section 1600 on a trigger ring body easily pass through the wall during pivoting of the actuation member 3000.

The actuation protrusion 3600 may further comprise a side wall 3660 configured to stop the movement of the trigger ring 1000. The side wall 3660 may be in the form of a flat surface or a curved surface or a combination of a flat surface and a curved surface. As shown in FIG. 11, an angle β between the side wall 3660 and the outer peripheral wall 3640 is greater than 60°, and the angle β is preferably in a range of 60° to 80°, preferably in a range of 65° to 70°, and particularly preferably in a range of 68° to 75°. With the arrangement of the side wall 3660 and/or the setting of the angle β, the side wall 3660 can make the protruding section 1600 on the trigger ring body not easily pass through the wall during the pivoting of the actuation member 3000.

In a preferred implementation, a length or an arc length of at least one side wall 3620 of the actuation protrusion 3600 for guidance on a horizontal projection plane is greater than or equal to a length or an arc length of at least one side wall 3660 of the actuation protrusion 3600 for stopping on the horizontal projection plane. This facilitates smooth movement of the protruding section 1600 between the side wall 3620 and the side wall 3660.

The actuation member 3000 comprises one or more ribs 3400 arranged on the inner wall of the actuation member body 3200. The ribs 3400 may be inserted into the grooves 2800 on the outer wall of the delivery tube holder 2000, so as to drive the delivery tube holder 2000 to pivot together.

In the present disclosure, the trigger assembly may be composed of, for example, the trigger ring 1000, the delivery tube holder 2000, and the actuation member 3000 as described above, and switching of the trigger assembly between the locked position and the unlocked position is implemented by means of an interaction between the three components. Two locked positions of a trigger assembly according to an implementation of the present disclosure are described in detail below with reference to FIGS. 13 to 18. A first locked position of the trigger assembly according to an implementation of the present disclosure is described in detail with reference to FIGS. 13 to 15, and FIGS. 16 to 18 show a second locked position of the trigger assembly. In addition, the unlocked position of the trigger assembly is described in detail by virtue of FIGS. 19 to 21. As mentioned above, the actuation member 3000 is sleeved on the delivery tube holder 2000, so that part of the structure of the delivery tube holder 2000 is hidden thereby. Therefore, for the sake of clarity, part of the structure of the actuation member 3000 is omitted in FIGS. 13 to 21, and only a part 3200′ of the actuation member body thereof is shown. However, it should be understood that the actual structure of the actuation member 3000 should be constructed as shown with reference to FIGS. 9 to 12. Herein, the “first locked position” and the “second locked position” are only distinguished for clear explanation, and do not represent a sequential relationship therebetween.

The trigger assembly in the first locked position is shown in FIGS. 13 to 15.

Herein, the first locked position, for example, corresponds to a pre-trigger position of the atomizer, that is, when the housing 8000 of the atomizer is rotated, the delivery tube holder 2000 moves downwardly along a spiral surface (preferably moves downwardly while rotating) to a position where the locking protrusion portion 1100 stops the locking notch 2100. In this pre-trigger position, the actuation member 3000 is sleeved on the delivery tube holder 2000 to restrict pivoting thereof, and the actuation protrusion 3600 is not in contact with the protruding section 1600 in this case.

In the first locked position, the first locking surface 1110 of the locking protrusion 1100 abuts against the second locking surface 2110 of the locking notch 2100, so as to implement stop of the trigger ring 1000 and the delivery tube holder 2000.

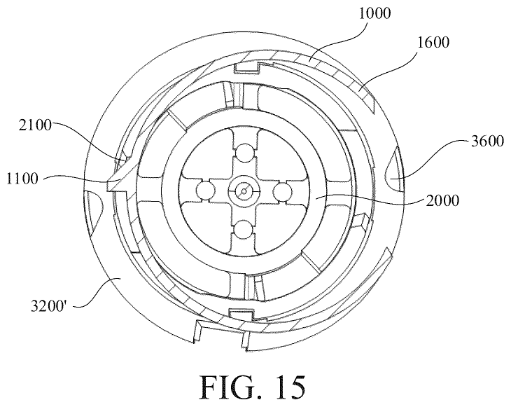

As can be seen from FIG. 15, in the first locked position, the trigger ring 1000 is arranged non-coaxially with respect to the delivery tube holder 2000 and the actuation member 3000 (with a part 3200′ of the actuation member body shown herein), that is, the trigger ring, the delivery tube holder, and the actuation member are arranged eccentrically. Therefore, in this first locked position, the trigger ring 1000 is pressed against both the delivery tube holder 2000 and the actuation member 3000. In this case, the actuation member 3000 does not apply a thrust to the trigger ring 1000.

Herein, in order to switch the trigger assembly from the locked position to the unlocked position, two unlocking modes may be employed. In the first mode, for example, a horizontal thrust is applied to the trigger ring 1000 until the delivery tube holder 2000 can axially move through the trigger ring 1000. The horizontal thrust may be indirectly applied to the trigger ring 1000 by manually pressing the push switch, or may be directly applied to the trigger ring 1000 manually. In the second mode, the actuation member 3000 is rotated to make the trigger assembly enter the second locked position, and then the actuation member 3000 is further rotated to make the trigger assembly enter the unlocked position. It can be seen that the unlocking of the trigger assembly can be implemented not only by, for example, the horizontal thrust, but also by a pivoting force. The two unlocking modes are particularly advantageous. This solves the problem of jamming or misoperation that is likely to occur when only one unlocking mode is provided, and provides more unlocking possibilities for the user, so that the user can gain the freedom to choose how to unlock the atomizer.

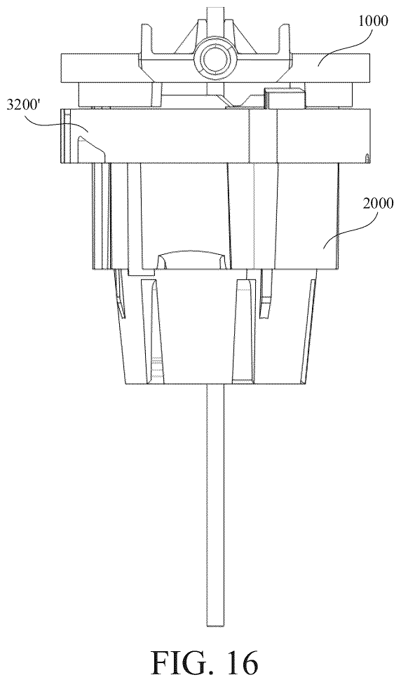

The trigger assembly in the second locked position is shown in FIGS. 16 to 18.

The trigger assembly can enter the second locked position from the first locked position by pivoting the actuation member 3000. During this process, the delivery tube holder 2000 pivots with the actuation member 3000.

In this second locked position, the actuation protrusion 3600 is in contact with the protruding section 1600 and can start to apply a thrust to the protruding section 1600 during further pivoting until the first locking surface 1110 abutting against the second locking surface 2110 of the locking notch 2100 passes across the second locking surface 2110, so that the trigger ring 1000 no longer stops the delivery tube holder 2000.

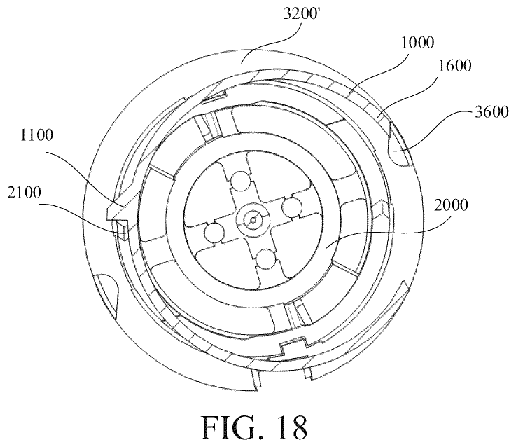

As can be seen from FIG. 18, in the second locked position, the trigger ring 1000 is still arranged non-coaxially with respect to the delivery tube holder 2000 and the actuation member 3000 (with a part 3200′ of the actuation member body shown herein), that is, the trigger ring, the delivery tube holder, and the actuation member are arranged eccentrically. Therefore, in this second locked position, the trigger ring 1000 is still pressed against both the delivery tube holder 2000 and the actuation member 3000. In this case, the actuation member 3000 starts to apply a thrust to the trigger ring 1000.

The trigger assembly in the unlocked position is shown in FIGS. 19 to 21.

The trigger assembly can enter the unlocked position from the second locked position by further pivoting the actuation member 3000. During this process, the actuation protrusion 3600 is actuated to drive the trigger ring 1000 until the trigger ring enters the unlocked position.

In this unlocked position, the actuation protrusion 3600 has pushed the protruding section 1600 back horizontally, so that the delivery tube holder 2000 can move axially through the trigger ring 1000. Therefore, the trigger ring 1000 is pressed only against the actuation member 3000, but not against the delivery tube holder 2000. In other words, in this unlocked position, the trigger ring 1000 only acts on the actuation member 3000, and no longer acts on the delivery tube holder 2000.

It should be understood that once the trigger assembly leaves the locked position, the trigger assembly enters the unlocked position. When the first locking surface 1110 is no longer in contact with the second locking surface 2110, the delivery tube holder 2000 can move in an axial direction relative to the trigger ring 1000.

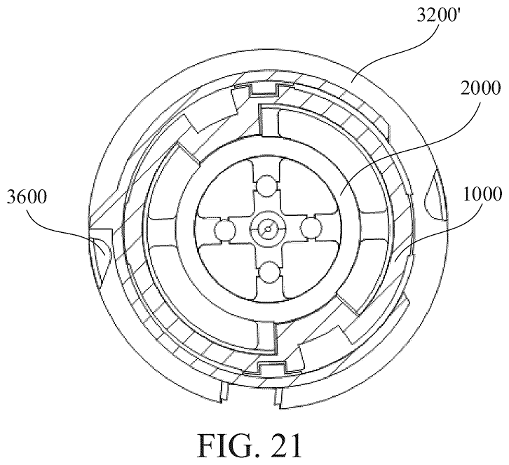

As can be seen from FIG. 21, in this unlocked position, the trigger ring 1000 is arranged coaxially with respect to the delivery tube holder 2000, that is, the trigger ring and the delivery tube holder are no longer eccentric to each other. In this case, if the outer diameter of the delivery tube holder 2000 is less than or equal to the inner diameter of the trigger ring 1000, the delivery tube holder 2000 can pass through the trigger ring 1000 to reach the unlocked position.

In order to urge the delivery tube holder 2000 to pass through the trigger ring 1000, a thrust mechanism, such as a spring 5000, may be arranged below the delivery tube holder 2000. When the spring 5000 is provided, the unlocked position is a transition state, because once the first locking surface 1110 slides past the second locking surface 2110, the delivery tube holder 2000 moves upwardly under the action of the spring 5000. When no spring is provided, the unlocked position is in a relatively stable state, because even if the first locking surface 1110 slides past the second locking surface 2110, the delivery tube holder 2000 is not forced immediately to move upwardly. In this case, other passive operation structures such as a button may be considered.

No matter whether it is due to active pressing by the spring or passive pushing by other alternative structures such as a button, the trigger assembly is in an unlocked state in this case. The unlocked state may correspond to an initial state of the atomizer, that is, a state in which no operation is performed on the atomizer. When the delivery tube holder 2000 or the actuation member 3000 rotates with the housing 8000 and the actuation member 3000 from the initial state, the delivery tube holder 2000 pivots downwardly with respect to the trigger ring 1000 under the action of the spiral surface 2600 until the first locking surface 1110 abuts against the second locking surface 2110, reaching the first locked position shown in FIG. 13, that is, the pre-trigger state of the atomizer. In this case, two different operations may be performed. The first operation is to apply a substantially horizontal thrust to the trigger ring 1000, so that the trigger assembly can leave the locked position to reach the unlocked position shown in FIG. 19. The second operation is to pivot the delivery tube holder 2000 or the actuation member 3000 to reach the second locked position shown in FIG. 16, and continue to pivot the delivery tube holder 2000 or the actuation member 3000 to reach the unlocked position shown in FIG. 19 under the driving of the actuation protrusion 3500 to the trigger ring 1000, thus completing a stroke cycle.

It may be understood that the stroke cycle of the trigger assembly may correspond to a full-circle rotation or a part-circle rotation of the trigger ring 1000, the delivery tube holder 2000 and/or the actuation member 3000, depending on the corresponding number and arrangements of locking protrusion portions 1100, locking notches 2100, actuation protrusions 3600 and protruding sections 1600 that are provided on the trigger ring 1000, the delivery tube holder 2000 and/or the actuation member 3000.

Finally, an atomizer according to an example of the present disclosure is described with reference to FIGS. 22 to 24. In order to control or prevent triggering of the atomizer, the atomizer comprises the trigger assembly as described above. Herein, the delivery tube holder 2000 and/or the actuation member 3000 is in the form of a housing of the atomizer, or the delivery tube holder 2000 and/or the actuation member 3000 is connected to the housing of the atomizer, so that the delivery tube holder 2000 can rotate or spirally move as the housing rotates. The trigger ring 1000 is in the form of a switch button of the atomizer, or the trigger ring 1000 is connected to the switch button of the atomizer, so that the trigger ring 1000 can move as the switch button is pressed.

When the atomizer is in an initial position, the trigger assembly is located in the unlocked position, or when the atomizer is in a pre-trigger position, the trigger assembly is located in the locked position. Therefore, the delivery tube holder 2000 can rotate in the unlocked position, such that the atomizer moves from the initial position to the pre-trigger position, and the trigger ring 1000 can be pressed in the locked position or be pushed by the actuation protrusion 1600 to move, for example, moving substantially horizontally, such that the atomizer moves from the pre-trigger position back to the initial position.

In addition to the trigger assembly, the atomizer may further comprise other components, such as a housing 8000, a push switch 6000, a container 4000 configured to contain a liquid to be atomized, a spring 5000 for applying a thrust to the delivery tube holder 2000, a mouthpiece 7000 (which can be put in the user's mouth during use), a spraying assembly 9000 configured to perform atomization and spray an atomized liquid, and a dust cover 11000.

It should be understood that the internal structure and the components of the atomizer shown in the figures are exemplary rather than limiting structures, provided that a function of preventing misoperation or accidental triggering of the atomizer can be achieved herein. Therefore, the atomizer comprising the trigger assembly capable of achieving this function can have any structure and components, which are not limited to the components and arrangements in the above figures. In addition, the above operation mode of the atomizer is also exemplary, but not restrictive. When a component of the atomizer is omitted or replaced, the operation mode of the atomizer can be changed accordingly to meet use requirements.

The above are merely embodiments or examples of the present disclosure and thus do not limit the patent scope of the present disclosure, and any equivalent structural changes made under the concept of the present disclosure by utilizing the contents of the description and the accompanying drawings of the present disclosure, or direct/indirect applications in other related technical fields shall fall within the scope of protection of the present disclosure. Various elements in the embodiments or examples may be omitted or substituted by equivalent elements thereof. In addition, the steps may be performed in an order different from that described in the present disclosure. Further, various elements in the embodiments or examples may be combined in various ways. It is important that, as the technology evolves, many elements described herein may be replaced with equivalent elements that appear after the present disclosure.

What is claimed is:

1.-39. (canceled)

40. A trigger assembly for an atomizer, the trigger assembly comprising a trigger ring and a delivery tube holder,
wherein the trigger assembly has a locked position and an unlocked position,
in the locked position, the trigger ring stops axial movement of the delivery tube holder, and in the unlocked position, the delivery tube holder is movable in an axial direction relative to the triggering, and
wherein
the trigger assembly further comprises an actuation member, the actuation member being actuated to drive the delivery tube holder to pivot and drive the trigger ring to move, so that the trigger assembly is switched between the locked position and the unlocked position.

41. The trigger assembly according to claim 40, wherein
the actuation member comprises an actuation member body and an actuation protrusion axially protruding from the actuation member body, the actuation protrusion being configured to drive the trigger ring to move from the locked position to the unlocked position, and/or to drive the trigger ring to move from the unlocked position to the locked position.

42. The trigger assembly according to claim 41, wherein
the actuation member comprises a plurality of actuation protrusions, wherein
at least one actuation protrusion of the plurality of actuation protrusions is configured to drive the trigger ring to move from the locked position to the unlocked position, and
at least another actuation protrusion of the plurality of actuation protrusions is configured to drive the trigger ring to move from the unlocked position to the locked position.

43. The trigger assembly according to claim 41, wherein
the actuation member comprises a plurality of actuation protrusions, wherein
each actuation protrusion of the plurality of actuation protrusions is capable of driving the trigger ring to move from the locked position to the unlocked position, and is capable of driving the trigger ring to move from the unlocked position to the locked position.

44. The trigger assembly according to claim 41, wherein
the plurality of actuation protrusions are arranged at equal angular intervals on the actuation member body in a circumferential direction of the actuation member.

45. The trigger assembly according to claim 41, wherein
at least one side wall of the actuation protrusion is configured to guide movement of the trigger ring;
an angle between the at least one side wall of the actuation protrusion for guidance and the outer peripheral wall is less than 45°, and the angle is preferably in a range of 25° to 45°, preferably in a range of 30° to 40°, and particularly preferably in a range of 31° to 35°.

46. The trigger assembly according to claim 41, wherein
at least one side wall of the actuation protrusion is configured to stop the movement of the trigger ring;
an angle between the at least one side wall of the actuation protrusion for stopping and the outer peripheral wall is greater than 60°, and the angle is preferably in a range of 60° to 80°, preferably in a range of 65° to 70°, and particularly preferably in a range of 68° to 75°.

47. The trigger assembly according to claim 41, wherein
the trigger ring comprises a ring body and a protruding section axially protruding from the ring body;
a side surface of the actuation protrusion is capable of abutting against a side surface of the protruding section to move the trigger ring from the locked position to the unlocked position and/or from the unlocked position to the locked position.

48. The trigger assembly according to claim 40, wherein
in the locked position, the trigger ring is pressed against the delivery tube holder and the actuation member, and
in the unlocked position, the trigger ring is pressed against only the actuation member.

49. The trigger assembly according to claim 40, wherein
in the locked position, the trigger ring is arranged non-coaxially with respect to the delivery tube holder and the actuation member, and
in the unlocked position, the trigger ring is arranged coaxially with respect to the delivery tube holder and the actuation member.

50. The trigger assembly according to claim 40, wherein
the trigger ring comprises a ring body and a first locking portion arranged on the ring body, and
the delivery tube holder comprises a tube holder body and a second locking portion arranged on the tube holder body.

51. The trigger assembly according to claim 50, wherein
in the locked position, the first locking portion abuts against the second locking portion, so that the trigger ring stops the axial movement of the delivery tube holder; or
in the locked position, the first locking portion abuts against the second locking portion, so that the trigger ring and the delivery tube holder stop each other.

52. The trigger assembly according to claim 51, wherein the trigger ring and the delivery tube holder stopping each other consists in that
the trigger ring prevents a vertical movement of the delivery tube holder; and
the delivery tube holder prevents a horizontal movement of the trigger ring.

53. The trigger assembly according to claim 52, wherein
the first locking portion is in the form of a locking protrusion extending out from at least one side of the trigger ring, and
the second locking portion is in the form of a locking notch recessed on at least one side of the tube holder body; or
the first locking portion is in the form of a locking notch recessed on at least one side of the trigger ring, and
the second locking portion is in the form of a locking protrusion extending out from at least one side of the tube holder body.

54. The trigger assembly according to claim 53, wherein only one locking protrusion is provided on at least one side of the trigger ring, and a plurality of locking notches are provided on at least one side of the delivery tube holder.

55. The trigger assembly according to claim 53, wherein the plurality of locking notches are arranged at equal angular intervals in a circumferential direction on a side of the delivery tube holder that mates with the locking protrusion.

56. The trigger assembly according to claim 50, wherein
the first locking portion is in the form of a first locking surface, and the second locking portion is in the form of a second locking surface;
at least one of the first locking surface and the second locking surface is in the form of a flat inclined surface, and the inclined surface is inclined with respect to both a horizontal direction and a vertical direction.

57. An atomizer, comprising a trigger assembly, for triggering the atomizer to spray an atomized liquid, wherein
the trigger assembly comprising a trigger ring and a delivery tube holder,
wherein the trigger assembly has a locked position and an unlocked position,
in the locked position, the trigger ring stops axial movement of the delivery tube holder, and in the unlocked position, the delivery tube holder is movable in an axial direction relative to the trigger ring, and
wherein
the trigger assembly further comprises an actuation member, the actuation member being actuated to drive the delivery tube holder to pivot and drive the trigger ring to move, so that the trigger assembly is switched between the locked position and the unlocked position.

58. The atomizer according to claim 57, wherein
the actuation member and/or the delivery tube holder is in the form of a housing of the atomizer; or
at least one of the actuation member and the delivery tube holder is connected to the housing of the atomizer, so that the actuation member and the delivery tube holder are rotatable as the housing rotates.

59. The atomizer according to claim 57, wherein
the trigger ring is in the form of a switch button of the atomizer; or
the trigger ring is connected to the switch button of the atomizer, so that the trigger ring is movable as the switch button is pressed.
